(19) United States
(12) Patent Application Publication
(10) Pub. No.: US 2008/0038721 A1
(43) Pub. Date: Feb. 14, 2008

(54) Sequence Capable Of Accelerating Gene Expression At Moderately Low Temperature

(76) Inventor: Jun Fujita, Kyoto-shi (JP)

Correspondence Address: BIRCH STEWART KOLASCH & BIRCH, PO BOX 747, FALLS CHURCH, VA, 22040-0747, US

(21) Appl. No.: 10/592,520

(22) PCT Filed: Mar. 1, 2005
(86) PCT No.: PCT/JP05/03385

§ 371 Date: Sep. 12, 2006

(30) Foreign Application Priority Data
Mar. 23, 2004 (JP) 2004-084987

(51) Int. Cl. C12P 21/04; C12N 15/00; C12N 15/11; C12Q 1/68; C12N 5/06

(52) U.S. Cl. 435/6; 435/320.1; 435/325; 435/69.1; 536/24.2

(57) ABSTRACT

It is an object of the present invention to provide a method for increasing protein yield in recombinant DNA method using mammalian cell. When a mild low temperature transcription control element comprising at least one nucleotide sequence:
x₁ccccX₆x₇X₈

wherein x₁ represents t, g, or a; x₆ represents g, a, or c; x₇ represents c, a or t; x₈ represents c, a or t, or a nucleotide sequence complementary to the nucleotide sequence is used as an enhancer and mammalian cells are cultivated at a mild low temperature, the transcription of a desired protein is accelerated. When mild low temperature translation control element of SEQ ID NO: 23 is placed 5′ to a coding sequence and 3′ to a promoter, and mammalian cells are cultivated at a mild low temperature, the translation of a desired protein is accelerated.

### TECHNICAL FIELD

The present invention relates to mild low temperature response elements accelerating the transcription or the translation of a gene at mild low temperatures.

### BACKGROUND ART

Various pharmaceuticals and assay reagents such as monoclonal antibodies, growth factors and clotting factors have been produced by biotechnology.

Generally, at a low temperature of 10-15° C., correct folding of heterologous recombinant protein is promoted and degradation of the protein by proteases is inhibited (Baneyx, F., In vitro folding of recombinant proteins in Escherichia coli. In Manual of industrial microbiology and biotechnology, 2nd ed., (Demain, A. L. Davis, J. E., Altas, R. M., et al., Eds.) ASM Press, Washingtonn D.C., pp. 551-1999). Accordingly, when recombinant proteins are produced by bacteria, cultivation at low temperatures below 20° C. is effected, instead of at 37° C. In this case, since protein synthesis is generally lowered at low temperature, possible low yield of a desired protein may occur. This problem is solved by using the promoter of cold shock protein CspA gene and 5′ untranslated region (UTR) of CspA mRNA in an expression vector of a desired protein (Baneyx, F. and Mujacic, M., Cold-inducible promoters for heterologous protein expression, Methods in Molecular Biology, 205, 1-18, 2001).

However, posttranslational modification in bacteria is significantly different from that in mammalian cells. Especially for pharmaceuticals, glycosylation state is important for therapeutic effects. Therefore, mammalian cells, particularly Chinese hamster cell lines, are used for producing protein pharmaceuticals. It is necessary to increase protein yield in order to reduce production cost with the cell cultivation. When usual cultivation at 37° C. is effected, there is a disadvantage that, since cells die after rapid growth, the term during which cells survive and produce the desired protein is short. Therefore, an attempt to restrain cell growth and have cells survive for a longer time period is effected by the addition of cell growth inhibitors, the expression of cell-cycle inhibitory gene p27, the cultivation at mild low temperatures (30-32° C.), etc. (see Schatz S M et al., Biotechnol. Bioeng, November 2003, 433-438).

Among them, the cultivation at mild low temperature has the characteristics as follows:

1) Cells survive and produce recombinant proteins for a longer time period than the cultivation at 37° C.;

2) The activity of proteolytic enzyme is lowered to reduce the degradation of desired proteins;

3) Glycosylation and isoprotein patterns are similar to those cultivated at 37° C., and sialylation is rather better than that cultivated at 37° C.;

4) A possible improvement in correct folding of desired proteins as in bacteria

(Kaufmann H, Mazur X, Marone R, Bailey J E, Fussengger M., Biotech Bioeng., Mar. 20, 2001, 72(6):592-602; Yoon S K, Song J Y, Lee G M., Biotech Bioeng., May 5, 2003, 82(3):289-98).

In regard to crucial yield of recombinant protein, since cell survive terms is extended, total yield is often increased. However, the specific productivity of protein per cell per unit time varies for unknown reasons, possibly depending on kinds of cell, kinds of the desired protein, and others. There are many cases in which the specific productivity is rather lower than that at 37° C., which is problematic (Kaufmann H, Mazur X, Marone R, Bailey J E, Fussenegger M., Biotech Bioeng., Mar. 20, 2001, 72(6):592-602; Yoon S K, Song J Y, Lee G M., Biotech Bioeng., May 5, 2003 82(3):289-98). Since gene expression control mechanism is different from that in bacteria, cold shock protein CspA promoter and 5′ untranslated region (UTR) of CspA mRNA in bacteria cannot be used in mammalian cells.

It is an object of the present invention to provide a method for increasing protein production and total yield per cell per unit time at low temperature.

### DISCLOSURE OF INVENTION

The inventor of the present application previously found and reported the cold shock proteins Cirp (cold inducible RNA-binding protein) the genomic DNA sequence of which is shown as SEQ ID No:1, and Rbm3 (RNA-binding motif protein 3) (Fujita J., Cold shock response in mammalian cells, J. Mol Microbiol. Biotechnol., Nov. 1, 1999 (2):243-55).

Now, the present invention has been made based on that the gene expression control sequences capable of accelerating transcription are found on 5′ side to the promoter of these protein genes.

That is, the present invention relates to a mild low temperature transcription control element comprising nucleotide sequence:
X₁ccccX₆X₇X₈

wherein x₁ represents t, g, or a; x₆ represents g, a or c; x₇ represents c, a, or t; and x₈ represents c, a and t, or a nucleotide sequence complementary to the sequence.

The term “mild low temperature” herein refers to a temperature of 15-36° C., preferably a temperature of 30-34° C. and most preferably a temperature of 32° C. The term “mild low temperature transcription control element” refers to a nucleotide sequence capable of accelerating the transcription of a gene specifically at mild low temperatures. The mild low temperature transcription control element of the present invention functions as a mild low temperature specific enhancer in the transcription of a gene.

The mild low temperature transcription control element of the present invention can exhibit its function when it is used alone. However, it is preferable to use it in connection of 2-50, preferably 2-10 of the same or different elements. When the elements are used in connection, the same or different elements may be directly connected, or may be connected via any number and kind of nucleotides. The connected mild low temperature transcription control elements are referred to as “mild low temperature transcription control enhancer” in the present specification. The mild low temperature transcription control element and the mild low temperature transcription control enhancer of the present invention have no relation to the distance from a promoter.

The present invention relates to a vector comprising the mild low temperature transcription control element or the mild low temperature transcription control enhancer described above.

The present invention relates to mammalian cells transformed by the vector.

The present invention relates to a method for screening a mild low temperature transcription control element comprising:

1) providing a gene capable of accelerating transcription at a mild low temperature, particularly preferably at a cultivation temperature of 32° C.;

2) cloning a part of the gene ranging from the 1.5-kilobase 5′ to transcription initiation site to the 3′ end of the first exon of the gene;

3) inserting the cloned nucleotide sequence with an appropriate promoter into a vector expressing a reporter gene;

4) transforming mammalian cells with the said vector;

5) cultivating the cells at a mild low temperature or 37° C. to express the reporter gene to identify the nucleotide sequence capable of accelerating the transcription of the reporter gene at a mild low temperature; and

6) shortening the nucleotide sequence to be inserted into the vector for the nucleotide sequence capable of accelerating the transcription of the reporter gene to identify the nucleotide sequence necessary for accelerating the transcription of the reporter gene at the mild low temperature.

The present invention relates to a method for improving a mild low temperature transcription control element comprising:

1) synthesizing a mutated nucleotide sequence in which one or two nucleotides in a mild low temperature transcription control element are substituted with other nucleotides;

2) incorporating the mutated nucleotide sequence with an appropriate promoter into a vector expressing a reporter gene;

3) transforming mammalian cells with the said vector;

4) cultivating the cells at a mild low temperature or 37° C. to express the reporter gene to identify the nucleotide sequence capable of accelerating the transcription of the reporter gene at the mild low temperature.

The inventor has further found a gene expression control sequence capable of accelerating translation at mild low temperature in the 5′ untranslated region (UTR) of the Cirp gene mRNA.

That is, the present invention relates to a mild low temperature translation control element comprising the nucleotide sequence:
actcg cgcct tagga agctt gggtg tgtgt ggcgc gctgt cttcc cgctc gcgtc aggga cctgc ccgac tcagc ggctg cc (SEQ ID NO:23) placed 5′ to a coding sequence and 3′ to a promoter.

The term “mild low temperature translation control element” herein refers to an element accelerating translation of transcribed mRNA specifically at mild low temperature. When the mild low temperature translation control element is transcribed, it forms the 5′untranlated region (UTR) of mRNA.

The term “mild low temperature response element” herein means the mild low temperature transcription control element and/or the mild low temperature translation control element.

### EFFECT OF THE PRESENT INVENTION

The present invention can accelerates the transcription and the translation of a gene at mild low temperatures.

### BRIEF DESCRIPTION OF DRAWINGS

FIG. 1 is a schematic view representing pCAT3 basic vector. MCS represents a multi-cloning site. CAT represents a coding sequence of chloramphenicol acetyltransferase. PA represents a polyadenylation signal derived from SV40 virus. Amp^{r} represents ampicillin resistance gene.

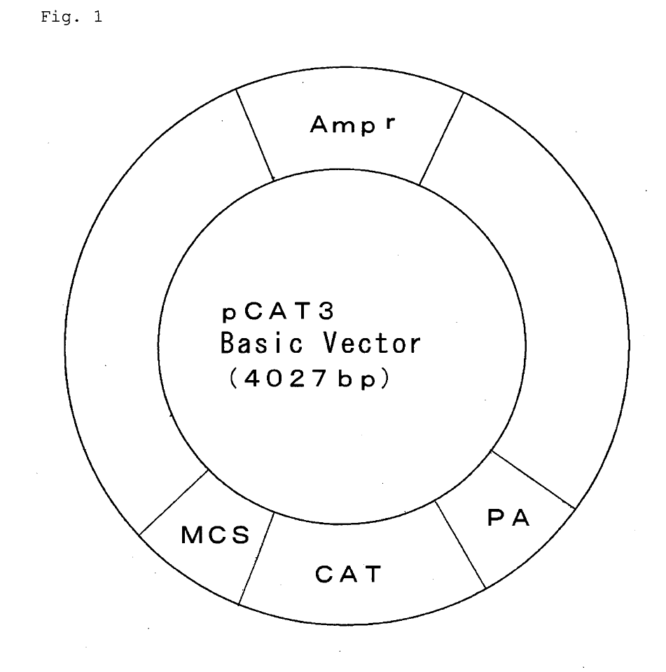

FIG. 2 is a schematic view representing pCAT3 promoter vector. MCS represents a multi-cloning site. SV40 promoter represents a promoter derived from SV40 virus. CAT represents a coding sequence of chloramphenicol acetyltransferase. PA represents a polyadenylation signal derived from SV40 virus. Ampᵣ represents an ampicillin resistance gene.

FIG. 3 is a schematic view representing pcDNA 3.1(+) vector (Invitrogen). MCS represents a multi-cloning site. Firefly luciferase encoding sequence is inserted between EcoRV and XbaI of the multi-cloning site. Complementary DNA (cDNA) for 5′ untranslated region (UTR) of Cirp mRNA is inserted between KpnI and BamHI of the multi-cloning site

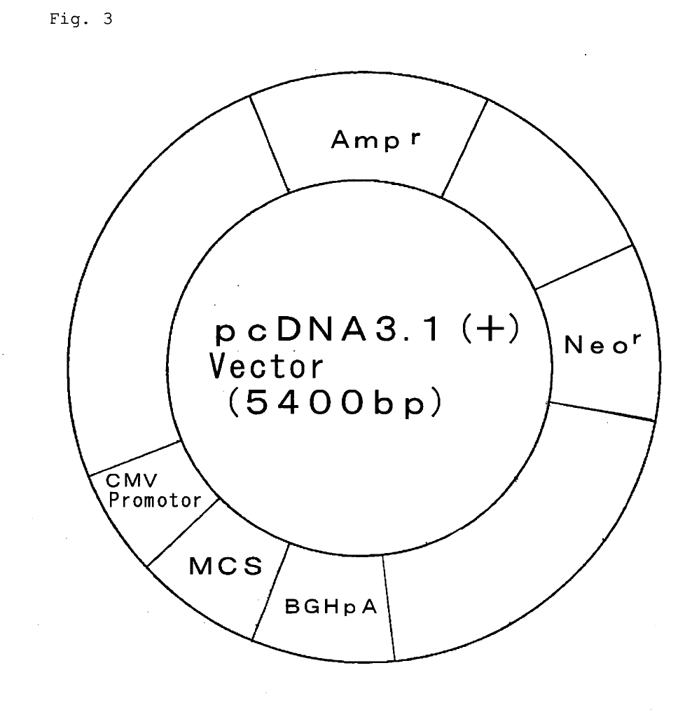

### BEST MODE FOR CARRYING OUT THE INVENTION

Examples of the mild low temperature transcription control element of the present invention include, but is not limited to, tcccc gcc (SEQ ID NO:2), gcccc gcc (SEQ ID NO:9), acccc gcc (SEQ ID NO:10), tcccc gtc (SEQ ID NO:11), tcccc gct (SEQ ID NO:12), tcccc gaa (SEQ ID NO:21), ggcgg gga (SEQ ID NO:22) and ggggg gga (SEQ ID NO:7)

(i) Preparation of Mild Low Temperature Response Element

The mild low temperature transcription control element, the mild low temperature transcription control enhancer and the mild low temperature translation control element of the present invention can be prepared, for example, via a solid synthesis method by phosphoroamidide method or via a solid synthesis method by hydrogen phosphonate method, using four kinds of deoxyribonucleotide with protective groups (see, for example, Oligonucleotide Synthesis: A Practical Approach, IRL Press, 35-81,83-115 (1984); J. Org. Chem., 49, 2139 (1984); Tetrahedron Lett., 27, 469 (1986); Tetrahedron Lett.,22, 1859-1862; Tetrahedron Lett., 21, 719-722; J. Am. Chem. Soc., 103, 3185-3191 (1981). Nowadays they can be easily synthesized with commercially available DNA synthesizers (for example, manufactured by Applied Biosystem).

(ii) Promoter to be Used

Prokaryote promoters, eukaryote promoters and virus promoters can be used as promoters. Mammalian promoters and virus promoters are preferred. Examples of preferred promoters include mouse thymidine kinase promoter (TK), cytomegalovirus (CMV) promoter and SV40 promoter.

(iii) Coding Sequence

Genes to be expressed are not limited, and the nucleotide sequence encoding any protein can be used.

(iv) Vector to be Used

The mild low temperature transcription control element or the mild low temperature transcription control enhancer of the present invention, a promoter and the coding sequence encoding a protein to be expressed are linked together and inserted into a vector. The expression vector for prokaryote cell and eukaryote cell can be used. Examples of preferred vector include, but not limited to, PCMV (Invitrogen), pCAT3 promoter vector (Promega) and pSV2-neo vector.

(v) Host Cell to be Used

Host cells are transfected with the vector. It is preferred to use mammalian cells as host cell. Examples of preferred mammalian cell are human cell lines such as 293, 293T, U-20S, Huh-7, HeLa cell; monkey cell lines such as COS-7 and Vero cell; mouse cell lines such as NIH/3T3, Balb/3T3, B-6, Hepa-6 and Chinese hamster cell lines such as CHO.

The methods for the transfection include calcium phosphate method, lipofection method and electroporation method. For example, in the calcium phosphate method, calcium chloride solution containing DNA (vector) is added dropwise to phosphoric acid solution with stirring. Refined precipitation containing DNA-calcium phosphate is formed. When the solution is added to host cell, the precipitation of DNA-calcium phosphate enters into host cells via phagocytosis so that DNA is transfected into cells.

(vi) Cultivation Condition

The host cell is cultivated at a mild low temperature. It is unnecessary to use special medium as culture medium. Usual medium for mammal cell cultivation can be used. Typical example of the medium is Dulbecco's modified Eagle's medium (D-MEM) supplemented with 10% fetal calf serum (FCS) (Invitrogen).

The present invention relates to a method for screening a mild low temperature transcription control element comprising:

1) providing a gene capable of accelerating transcription at a mild low temperature, particularly preferably at a cultivation temperature of 32° C.;

2) cloning a part of the gene ranging from the 1.5-kilobase 5′ to transcription initiation site to the 3′ end of the first exon of the gene;

3) incorporating the cloned nucleotide sequence with an appropriate promoter into a vector expressing a reporter gene;

4) transforming mammalian cells with the said vector;

5) cultivating the cells at a mild low temperature or 37° C. to express the reporter gene to identify the nucleotide sequence capable of accelerating the transcription of the reporter gene at a mild low temperature; and

6) shortening the nucleotide sequence to be inserted into the vector for the nucleotide sequence capable of accelerating the transcription of the reporter gene to identify the nucleotide sequence necessary for accelerating the transcription of the reporter gene at the mild low temperature.

The gene capable of accelerating transcription at mild low temperature can be obtained by cultivating mammalian cells at 37° C. and at mild low temperature, preferably at 32° C. and applying DNA chip (microaray) or cDNA subtraction method (see, for example, Moody, D. E., Genomic techniques: An overview of methods for study of gene expression, J. Anim. Sci., 79(E. Suppl.): E128-E135. 2001) to the RNA extracted therefrom.

Prokaryote promoters, eukaryote promoters and virus promoters can be used as promoters. Mammalian promoters and virus promoters are preferred. Examples of preferred promoters include, but is not limited to, mouse thymidine kinase promoter (TK), cytomegalovirus (CMV) promoter and SV40 promoter.

Reporter gene is a gene encoding protein capable of being easily detected, and include, but is not limited to, luciferase gene and chloramphenicol acetyltransferase gene. Vectors for prokaryote cell and eukaryote cell can be used. Examples of preferred vector include, but is not limited to, pCMV (Invitrogen), pCAT3 promoter vector (Promega) and pSV2-neo vector.

Host cells are transfected with the vector. It is preferred to use mammalian cells as host cell. Examples of preferred mammalian cell are human cell lines such as 293, 293T, U-20S, Huh-7, HeLa cell; monkey cell lines such as COS-7 and Vero cell; mouse cell lines such as NIH/3T3, Balb/3T3, B-6, Hepa-6 and Chinese hamster such as CHO

The methods for the transfection include calcium phosphate method, lipofection method and electroporation method. For example, in the calcium phosphate method, calcium chloride solution containing DNA (vector) is added dropwise to phosphoric acid solution with stirring. Refined precipitation containing DNA-calcium phosphate is formed. When the solution is added to host cell, the precipitation of DNA-calcium phosphate enters into host cells via phagocytosis so that DNA is transfected into cells.

It is unnecessary to use special medium as culture medium. Usual medium for mammalian cell cultivation can be used. A typical example of medium is Dulbecco's modified Eagle's medium (D-MEM) supplemented with 10% fetal calf serum (FCS) (Invitrogen).

If the ratio of the amount of the reporter gene product expressed at a mild low temperature, preferably at 32° C., to that at 37° C. is more than 1, the nucleotide sequence can be an enhancer capable of accelerating the transcription of gene specifically at mild low temperature. The nucleotide sequence capable of accelerating the transcription of the gene at a mild low temperature is shortened to obtain a mild low temperature transcription control element of the present invention. That it is an enhancer can be confirmed by confirming that the nucleotide sequence complementary to the mild low temperature transcription control element can also accelerate the transcription of gene at a mild low temperature.

The present invention relates to a method for improving a mild low temperature transcription control element comprising:

1) synthesizing a mutated nucleotide sequence in which one or two nucleotides in a mild low temperature transcription control element are substituted with other nucleotide;

2) incorporating the mutated nucleotide sequence with an appropriate promoter into a vector expressing a reporter gene;

3) transforming mammalian cells with the said vector;

4) cultivating the cells at a mild low temperature or 37° C. to express the reporter gene to identify the nucleotide sequence capable of accelerating the transcription of the reporter gene at the mild low temperature.

The mutated nucleotide in which one or two nucleotides in the mild low temperature transcription control element are substituted with other nucleotides can be synthesized as described above.

Prokaryote promoters, eukaryote promoters and virus promoters can be used as promoters. Mammalian promoters and virus promoters are preferred. Examples of preferred promoters include mouse thymidine kinase promoter (TK), cytomegalovirus (CMV) promoter and SV40 promoter.

Reporter gene is a gene encoding protein capable of being easily detected, and includes luciferase gene chloramphenicol acetyltransferase gene. Vector for prokaryote cell and eukaryote cell can be used. Examples of preferred vector include, but is not limited to, pCMV (Invitrogen), pCAT3 promoter vector (Promega) and pSV2-neo vector.

Host cells are transfected with the vector. It is preferred to use mammalian cells as host cell. Examples of preferred mammalian cell are human cell lines such as 293, 293T, U-20S, Huh-7, HeLa cell; monkey cell lines such as COS-7 and Vero cell; mouse cell lines such as NIH/3T3, Balb/3T3, B-6, Hepa-6 and Chinese hamster such as CHO.

The methods for the transfection include calcium phosphate method, lipofection method and electroporation method. For example, in the calcium phosphate method, calcium chloride solution containing DNA (vector) is added dropwise to phosphoric acid solution with stirring. Refined precipitation containing DNA-calcium phosphate is formed. When the solution is added to host cell, the precipitation of DNA-calcium phosphate enters into host cells via phagocytosis so that DNA is transfected into cell.

It is unnecessary to use special medium as culture medium. Usual medium for mammalian cell cultivation can be used. Typical example of the medium is Dulbecco's modified Eagle's medium (D-MEM) supplemented with 10% fetal calf serum (FCS)(Invitrogen).

If the ratio of the amount of the reporter gene product expressed at a mild low temperature, preferably at 32° C., to that at 37° C. is more than 1, the sequence can be an enhancer capable of accelerating the transcription of gene specifically at a mild low temperature. That the sequence is an enhancer can be confirmed by confirming that the nucleotide sequence complementary to the mild low temperature transcription control element of the present invention also accelerates the transcription of gene at a mild low temperature.

The present invention also relates to a mild low temperature translation control element comprising the nucleotide sequence:
actcgcgcct taggaagctt gggtgtgtgt ggcgcgctgt cttcccgctc gcgtcaggga cctgcccgac tcagcggctg cc (SEQ ID NO:23) placed 5′ to a coding sequence and 3′ to a promoter.

Prokaryote promoters, eukaryote promoters and virus promoters can be used as promoter. Mammalian promoters and virus promoters are preferred. Examples of preferred promoters include mouse thymidine kinase (TK) promoter, cytomegalovirus (CMV) promoter and SV40 promoter. The mild low temperature transcription control element or mild low temperature transcription control enhancer of the present invention, and a promoter can be used together with the mild low temperature translation control element of the present invention.

Vectors for prokaryote cell and eukaryote cell can be used. Examples of preferred vector include, but is not limited to, pCMV (Invitrogen), pCAT3 promoter vector (Promega) and pSV2-neo vector.

Host cells are transfected with the vector. It is preferred to use mammalian cells as host cell. Examples of preferred mammalian cell are human cell lines such as 293, 293T, U-20S, Huh-7, HeLa cell; monkey cell lines such as COS-7 and Vero cell; mouse cell lines such as NIH/3T3, Balb/3T3, B-6, Hepa-6 and Chinese hamster such as CHO.

The methods for the transfection include calcium phosphate method, lipofection method and electroporation method. For example, in the calcium phosphate method, calcium chloride solution containing DNA (vector) is added dropwise to phosphoric acid solution with stirring. Refined precipitation containing DNA-calcium phosphate is formed. When the solution is added to host cell, the precipitation of DNA-calcium phosphate enters into host cell via phagocytosis so that DNA is transfected into cell.

It is unnecessary to use special medium as culture medium. Usual medium for mammalian cell cultivation can be used. Typical example of the medium is Dulbecco's modified Eagle's medium (D-MEM) supplemented with 10% fetal calf serum (Invitrogen).

### EXAMPLE

### Example 1

Assuming “a” of “actcgc . . . ” of the transcription start site of Cirp gene to be +1, the Cirp gene spanning the nucleotides −970 (970 nucleotides 5′ to the transcription start site) to +56 (within the first exon), and fragments thereof deleted on 5′ side (see Table 1) were inserted into the multi-cloning site of pCAT3-basic vector (Promega). The vector has chloramphenicol acetyltransferase (CAT) gene as the reporter gene.

Then 2.5-5.0 μg/35-mm dish of the plasmids were co-transfected with 0.5-1.0 μg/35-mm dish of CDM8-LacZ (which is β-galactosidase expression vector and used to correct for transfection efficiency) into human cell line 293.

The transfected cells were divided into two groups. One is cultivated at 32° C. and the other at 37° C. Medium was D-MEM supplemented with 10% FCS.

After 36 hours, cells were recovered by centrifugation and whole cell lysate was prepared. The amount of chloramphenicol acetyltransferase (CAT) protein produced at each temperature was determined using CAT ELISA kit (Roche Diagnostic), and β-galactosidase activity was determined usingβ-galactosidase assay kit (Stratagene). The transcription accelerating activity of each fragment is shown in Table 1. The transcription accelerating activity was calculated by dividing CAT protein mass by β-galactosidase activity.

TABLE 1
Transcription accelerating activity of
5′ side fragment of Cirp genome

| Cirp genome | | | |
|---|---|---|---|
| 5′side | CAT/Lac Z | | |
| fragment | 32° C. | 37° C. | 32° C./37° C. ratio |
| −970˜+56 | 2.007 ± 0.005 | 1.173 ± 0.004 | 1.711 |
| −910˜+56 | 2.804 ± 0.009 | 1.196 ± 0.008 | 2.345 |
| −850˜+56 | 2.518 ± 0.007 | 1.250 ± 0.010 | 2.015 |
| −790˜+56 | 2.014 ± 0.003 | 1.211 ± 0.009 | 1.664 |
| −720˜+56 | 2.586 ± 0.009 | 1.246 ± 0.007 | 2.076 |
| −620˜+56 | 2.008 ± 0.010 | 1.180 ± 0.007 | 1.702 |
| −520˜+56 | 2.251 ± 0.003 | 1.481 ± 0.011 | 1.520 |
| −430˜+56 | 2.298 ± 0.010 | 1.640 ± 0.008 | 1.402 |
| −340˜+56 | 2.729 ± 0.003 | 1.365 ± 0.004 | 2.000 |
| −220˜+56 | 0.045 ± 0.003 | 0.123 ± 0.002 | 0.366 |

The data at 24 hours after transfection was similar this result.

From these data, it is considered that basic promoter for gene transcription may exist at −220 to 0 of the genomic gene and an enhancer accelerating transcription in response to 32° C. may exist at −340 to −220.

### Example 2

Example 1 was repeated except that human cell line 293T, U-2 OS, or Huh-7, monkey cell line COS-7, or mouse cell line NIH/3T3, Balb/3T3, B-6 or Hepa-6 was used in place of human 293 as host cells, and similar results were obtained.

### Example 3

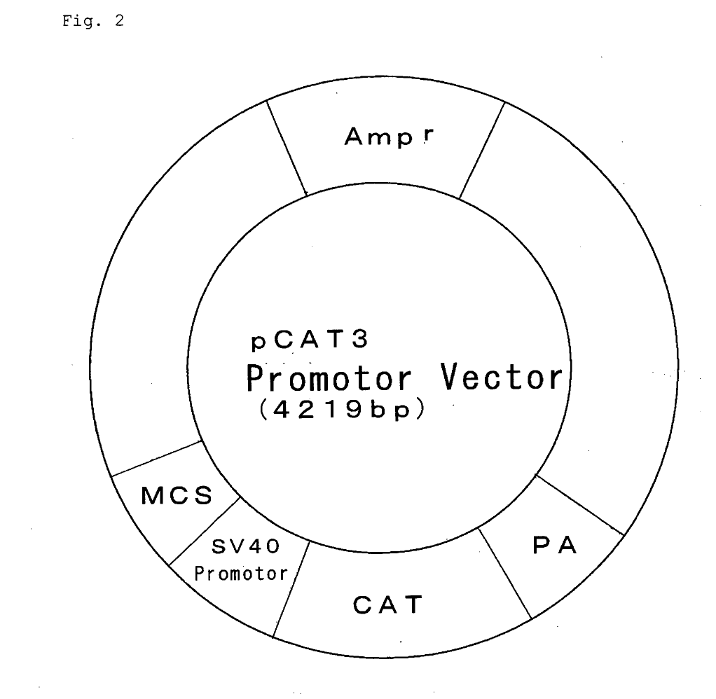

Various deletion mutants derived from the nucleotides −340 to −240 of the Cirp gene were prepared and inserted into the multi-cloning site of pCAT3 promoter vector (Promega) (FIG. 2). This vector has the promoter of SV40. As in Example 1, after the gene transfer by DNA transfection with calcium phosphate method using human 293 cells, transcription accelerating activity was measured at 32° C. pCAT 3 promoter vector containing no insert was used as a negative control (mock).

Data at 36 hours after transfection are shown. The transcription accelerating activity was calculated by dividing CAT protein mass by β-galactosidase activity (Table 2).

TABLE 2
Transcription accelerating activity of
5′ side fragment of Cirp genome

| Mutant with | | | |
|---|---|---|---|
| deletion in | | | |
| −340 to −220 | CAT/Lac Z | | |
| range | 32° C. | 37° C. | 32° C./37° C. ratio |
| mock | 0.226 ± 0.012 | 0.757 ± 0.010 | 0.299 |
| −340˜−220 | 2.629 ± 0.008 | 0.592 ± 0.004 | 4.441 |
| −340˜−290 | 4.064 ± 0.015 | 0.654 ± 0.010 | 6.215 |
| −290˜−220 | 2.419 ± 0.008 | 0.549 ± 0.008 | 4.406 |
| −310˜−260 | 2.089 ± 0.011 | 0.540 ± 0.003 | 3.869 |
| −340˜−310 | 7.697 ± 0.008 | 4.211 ± 0.031 | 1.828 |
| −310˜−290 | 8.234 ± 0.015 | 2.064 ± 0.017 | 3.990 |
| −290˜−260 | 10.217 ± 0.011 | 6.281 ± 0.030 | 1.627 |
| −260˜−220 | 9.681 ± 0.011 | 3.844 ± 0.011 | 2.519 |

It is considered from these results that there are enhancers in 2 genomic fragments, −310 to −290, and −260 to −220, which accelerate transcription in response to 32° C. Comparing the hucleotide sequence within the two fragments, common sequence of tcccc gcc (SEQ ID NO:2) has been found.

### Example 4

Three molecules of ten nucleotides containing tcccc gcc found above were connected in series to form a sequence ttccc cgccg ttccc cgccg ttccc cgccg (SEQ ID NO: 3). The sequence was inserted into the multi-cloning site of pCAT3 promoter vector as in Example 3. As a negative control, pCAT3 promoter vector containing no insert was used (mock). As positive controls, pCAT3 promoter vector containing Cirp genome from −340 to −220 as an enhancer and pCAT3 promoter vector containing SV 40 enhancer were used. The result obtained at 24 hours after transfection into 293 cells is shown in Table 3.

TABLE 3

| | CAT/Lac Z | | |
|---|---|---|---|
| | | | 32° C./37° C. |
| Enhancer | 32° C. | 37° C. | ratio |
| mock | 0.220 ± 0.003 | 0.677 ± 0.012 | 0.325 |
| SV40 | 0.908 ± 0.003 | 1.371 ± 0.010 | 0.662 |
| ttccccgccg | 6.622 ± 0.020 | 1.603 ± 0.009 | 4.132 |
| ttccccgccg | | | |
| ttccccgccg | | | |
| −320-−220 | 6.991 ± 0.031 | 1.461 ± 0.017 | 4.786 |

### Example 5

If a sequence is an enhancer, the reverse-directed sequence should be also effective. Three molecules of ten nucleotides containing cggcg gggaa (SEQ ID NO:4) which is a reverse directed sequence of a sequence comprising tcccc gcc, were connected in series to form a sequence cggcg gggaa cggcg gggaa cggcg gggaa (SEQ ID NO:5). The sequence was inserted into the multi-cloning site of pCAT3 promoter vector to carry out an experiment similar to Example 4.

In this case, the ratio of 32° C./37° C. was 3.83, demonstrating that the sequence is a mild low temperature transcription control enhancer.

### Example 6

A sequence tcccc gcctc cccgc c(SEQ ID NO:6 )obtained by connecting two molecules of 8 nucleotides tcccc gcc(SEQ ID NO:2) found in mouse Cirp genome in series was inserted into the multi-cloning site of pCAT3 promoter vector and an experiment similar to Example 4 was carried out. In this case, the ratio of 32° C./37° C. was 2.51, demonstrating that the sequence is a mild low temperature transcription control enhancer.

### Example 7

Three molecules of 8 nucleotides ggggg gga (SEQ ID NO:7), the sequence of which is found about 260 nucleotides 5′ to the transcription start site of human RBM genome (Gen Bank NT_{—}011568), were connected in series to form a sequence ggggg ggagg ggggg agggg ggga (SEQ ID NO:8). The sequence was inserted into the multi-cloning site of pCAT3 promoter vector and an experiment similar to Example 4 was carried out.

In this case, the ratio of 32° C./37° C. was 2.52, demonstrating that the sequence is a mild low temperature transcription control enhancer.

### Example 8

Three molecules of 10 nucleotides containing one or two base-substitutions (see the table below) in the sequence tcccc gcc (SEQ ID NO:2) found above were connected in series to form a sequence. The sequence was inserted into the same vector as in Example 4, and 293 cells were transfected by the vector. After 24 hours, CAT/LacZ was measured and the mild low transcription accelerating (enhancer) activity of respective mutants was shown in the table below, defining the ratio of 32° C./37° C. as 1 when pCAT3 promoter vector containing no insert was used.

TABLE 4

Low temperature transcription accelerating
(enhancer) activity of mutant with one base-
substitution

| | | Enhancer |
|---|---|---|
| Element | nucleotide sequence | activity |
| wild type | tcccc gcc (SEQ ID NO: 2) | 3.89 |
| mutant 1g | Gcccc gcc (SEQ ID NO: 9) | 3.60 |
| mutant 1a | Acccc gcc (SEQ ID NO: 10) | 3.74 |
| mutant 7t | tcccc gTc (SEQ ID NO: 11) | 3.63 |
| mutant 8t | tcccc gcT (SEQ ID NO: 12) | 4.08 |
| mutant 6a | tcccc Acc (SEQ ID NO: 13) | 2.14 |
| mutant 6c | tcccc Ccc (SEQ ID NO: 14) | 2.27 |
| control | | 1.00 |

TABLE 5

Low temperature transcription accelerating
(enhancer) activity of mutant with 2 base-
substitutions

| | | Enhancer |
|---|---|---|
| Element | nucleotide sequence | activity |
| wild type | tcccc gcc (SEQ ID NO: 2) | 3.89 |
| mutant 12 | GAccc gcc (SEQ ID NO: 15) | 1.76 |
| mutant 23 | tAAcc gcc (SEQ ID NO: 16) | 1.07 |
| mutant 34 | tcAAc gTc (SEQ ID NO: 17) | 1.47 |
| mutant 45 | tccAA gcc (SEQ ID NO: 18) | 0.35 |
| mutant 56 | tcccA Tcc (SEQ ID NO: 19) | 1.07 |
| mutant 67 | tcccc TAc (SEQ ID NO: 20) | 1.66 |
| mutant 78 | tcccc gAA (SEQ ID NO: 21) | 3.69 |
| control | | 1.00 |

The mutants with a replacement of the first base by G or A, a replacement of the seventh base by T, and a replacement of the eighth base by T exhibit the similar transcription accelerating activity to wild type sequence. Although the replacement of the sixth base by A or C lowers the activity compared with wild type sequence, they show significant activities. The mutant 78, having replacements of the seventh and eighth bases by AA, has the same degree of transcription accelerating activity as the wild type.

Accordingly, a sequence of tcccc gcc is a sequence showing transcription accelerating activity, and the sequence:

t(or g or a)ccccg(or a or c)c(or a or t)c(or a or t) can have transcription accelerating activity.

Since they are enhancers, the sequence complementary to them, for example, ggcgg gga (SEQ ID NO:22) complementary to tcccc gcc (SEQ ID NO:2) can also be used.

### Example 9

Firefly luciferase cDNA excised from pGL3-basic plasmid (Promega) was inserted into the multi-cloning site (between EcoRV and XbaI) of pcDNA3.1 (+)vector (Invitrogen) having cytomegalovirus (CMV) promoter. Then, a cDNA fragment corresponding to the +1 to +82 nucleotides (“a” of “act . . . ” of mouse Cirp transcription product (mRNA) being assumed to be +1) of Cirp mRNA, actcgcgcct taggaagctt gggtgtgtgt ggcgcgctgt cttcccgctc gcgtcaggga cctgcccgac tcagcggctg cc (SEQ ID NO:23) was inserted into the multi-cloning site (between KpnI and BamHI) of pcDNA3.1 (+) vector (Invitrogen). Control is a plasmid having no insert between KpnI and BamHI.

Then 2.5-5.0 μg/35-mm dish of these plasmids were co-transfected with 0.5-1.0 μg/35 mm-dish of pRLTK plasmid (which is renila lucuferase expression vector and used to amend efficiency of each transfection) into human cell line 293T which had been cultivated for one day. The transfected cells were divided into two groups. One is cultivated at 32° C. and the other at 37° C. Medium was D-MEM supplemented with 10% FCS.

After 36 hours, cells were recovered by centrifugation and whole cell lysate was prepared. The firefly and renila luciferase activities were determined using Dual-Luciferase Reporter kit (Promega). Translation accelerating activity of each fragment is shown in Table 6. The translation accelerating activity is shown as a value of firefly luciferase activity divided by renila luciferase activity.

TABLE 6
Translation accelerating activity of 5′ untranslated
region (UTR) of Cirp mRNA

| 5′ untranslated | | | |
|---|---|---|---|
| region (UTR) of | Firefly luciferase/Renila luciferase | | |
| Cirp mRNA | 32° C. | 37° C. | 32° C./37° C. ratio |
| absence | 3.52 | 6.08 | 0.58 |
| presence | 6.02 | 7.71 | 0.78 |

The above result shows that the amount of protein produced at 32° C. is increased by a factor of 1.7 when 5′UTR of Cirp mRNA is present in the vector compared with that in its absence, and that the amount is equal to the amount produced at 37° C. in cells transfected with plasmids containing no 5′UTR of Cirp mRNA.

1. A mild low temperature transcription control element consisting of a nucleotide sequence:
x₁ccccX₆x₇X₈
wherein x₁ represents t, g, or a; x₆ represents g, a, or c; x₇ represents c, a or t; x₈ represents c, a or t, or a nucleotide sequence complementary to the nucleotide sequence.

2. The mild low temperature transcription control element of claim 1, wherein the nucleotide sequence is tcccc gcc (SEQ ID NO:2).

3. The mild low temperature transcription control element of claim 1, wherein the nucleotide sequence is gcccc gcc (SEQ ID NO:9).

4. The mild low temperature transcription control element of claim 1, wherein the nucleotide sequence is acccc gcc (SEQ ID NO:10).

5. The mild low temperature transcription control element of claim 1, wherein the nucleotide sequence is tcccc gtc (SEQ ID NO:11).

6. The mild low temperature transcription control element of claim 1, wherein the nucleotide sequence is tcccc gct (SEQ ID NO:12).

7. The mild low temperature transcription control element of claim 1, wherein the nucleotide sequence is tcccc gaa (SEQ ID NO:21).

8. The mild low temperature transcription control element of claim 1, wherein the nucleotide sequence is ggcgg gga (SEQ ID NO:22).

9. The mild low temperature transcription control element of claim 1, wherein the nucleotide sequence is ggggg gga (SEQ ID NO:7).

10. A mild low temperature transcription control enhancer wherein identical or different mild low temperature transcription control elements of claim 1 are connected.

11. A mild low temperature transcription control enhancer wherein identical or different mild low temperature transcription control elements of claim 1 are connected via other nucleotide(s).

12. A vector comprising the mild low temperature transcription control element of any one of claims 1-9 or the mild low temperature transcription control enhancer of claim 10 or 11.

13. A mammalian cell transformed with the vector of claim 12.

14. A method for screening a mild low temperature transcription control element comprising:
1) providing a gene capable of accelerating transcription at a mild low temperature, particularly at a cultivation temperature of 32° C.;
2) cloning the range of from 1.5 kilo bases located on 5′ upstream of transcription initiation site of the gene to a first exon of the gene;
3) incorporating the cloned nucleotide sequence with an appropriate promoter into a vector expressing chloramphenicol actyltransferase (CAT) as a reporter gene;
4) transforming mammalian cells with the said vector;
5) cultivating the cells at a mild low temperature and 37° C., respectively, to express reporter gene to obtain a nucleotide sequence capable of accelerating the transcription of the reporter gene under mild low temperature; and
6) shortening the nucleotide sequence to be incorporated into the vector to identify the nucleotide sequence necessary for accelerating the transcription of the reporter gene at mild low temperature.

15. A method for improving a mild low temperature transcription control element comprising:
1) synthesizing a mutated nucleotide sequence in which one or two nucleotides in the mild low temperature transcription control element are substituted with other nucleotide;
2) incorporating the mutated nucleotide sequence with an appropriate promoter into a vector expressing chloramphenicol actyltransferase (CAT) as a reporter gene;
3) transforming mammalian cells with the said vector;
4) cultivating the cells at a mild low temperature and 37° C., respectively, to express the reporter gene to identify the nucleotide sequence necessary for accelerating the transcription of the reporter gene at a mild low temperature.

16. A mild low temperature translation control element comprising the nucleotide sequence: actcgcgcct taggaagctt gggtgtgtgt ggcgcgctgt cttcccgctc gcgtcaggga cctgcccgac tcagcggctg cc (SEQ ID NO:23) located 5′ to code sequence and 3′ to promoter.

17. A vector comprising the mild low temperature translation control element of claim 16.

18. A mammalian cell transformed with the vector of claim 17.


SEQUENCE LISTING (23 sequences)

SEQ ID NO: 1 - DNA, 2520 bp, mouse
ttgtcagcgcaagataggcaagtgctgcatagacattcaggcaaacacccatatacataaaattaaaatacacttttttgttttgttctctgtggtgtgtgtgtttaagaaggctaaggatgtagcttggtttagccactacttagaattctgcagtgaggggttaggggtgtggctcaaggatagaaatccttccatggcatgcttgagactctggggtctcttctagaaaatgcaggtacacattgaaaagagtggtgttttgccatctgtgtggtaatgtacacctggttggagatgggcagaaatcagggttcaaatggtcgttatacagcaggttgagatttgtaggaaaactgagttgggtgcaggaaatctagagaaaatcttgacattgtgattaatactgggatggaaattttagggctgatcttatggagtgtccttggggtccaaagaactgttaggcagcagttgtattgggaggtacttttggactagagtaattcggtggttttggtttcatgagcaagaacggagaagaggggcaagcaagatgactcagtggataaagctgcttgtggctaatcctgagtatgatccttaggacctacatgatggaaggagagactccatctgtcttctggcttcacaaatgcgcctcagttaaataactgtaattaaatattaaaagaaaaaaaagaacataaaagtgttgagtcgatgagtcaaggttggagccatatttgggagtctcattgaggtagcaaaagctctttgttccccgccgcaggacaaacgggtcgggaacgcggaagtgggtcagctccgcctccaaatcagctgtccccgcctcctagtcgctagcgggcggggggtgggggggaaaaatgcggccaggaaagattccagccaatcaaaacactcagcgcctccgcgcggactagggcaaagccaacggagtcccgcccctccaccgaggcgcagatgccgaatctaaggcgtcggattggtcagcgcggcgtggtgggcgggttaaggggcgtggctccaggagtggagtatatcaggcgggactcttgcgctccccctcgcactcgcgccttaggaagcttgggtgtgtgtggcgcgctgtcttcccgctcgcgtcagggacctgcccgactcagcggtgagggctctggcggcaccggctctgaggggcccggggcggcgggcggcctgtgactctcgggcgaggcggagccgcagcggcacgcccctgggacttgggggcgggcccgggttggagtccgccccgccggcggcgaggcgggcagcgggacactggggcctcacgcttgtcggagggcgtcctccaccgcgcgtcatccccgggccgaggccgaccttaacggtgggcttttttttttttttttttttttgtaaaatggccgccgcgatgctttgaaaagcttcgccggcgaaatggcgggactgccgagtgcccggatggaatggcgcagggtgcgcgcttggcgcatgcgctgctccgggggcggggcgctcctacctccaaagtccggttgtccgcagatgtggagctgtgtcgcgtgcgacccttttactccagctggctctacctcggttttctcttcccttgtggttccacgtggggaacaaaaaagcacatgtaggcttgaatgttgtccttttgtgccccaagtttattgcccccaaggtagactggtttcgtggtatagtggacttattttccaagttgtcggttgccaataatgactttgtctagataccacgcccctttcgctagccacgcctcttgggccaacctcctttctcccttgagtccatcgtgcaaaaacatttctctagtccttggctgagtaagcgaggcatcccacaactggcacacctccagacagtaccctaaaagcgtatttcagtagtgaagtttctctggcccaacaactctatgctagtgagggtaggcagtcactatcatttctgtccccaaccacacacatttgcaaagtgaccatgccattttgatcctaaccttgggattctctggccaggtgtaatcaagacctagaatcatttaaggttagcctcagctgtatgacaaagtattccatatgcctttgggatgataaaagcaaaaagagttccctcttgtgtagcccagaagggtattgaacttgctgtgcaattgtatttggttttggattctgttcctttgcctccaactcccacaggctaggcgagatgggtatttgttttgaaacagtctctcaaactgatccacagtgtcctttttctcattgatcttcccatctcggtgacttgaatggctggatgagaggtgtgaaagaaaccccacttgaagctgaggtttcccagtaagaccatggggtcctctccccccacccccatgcttcccccctagccccccccccccagtttctttgtgtactcctggttatcctggactctctttgcagaccaggctggcctcaaaac

SEQ ID NO: 2 - DNA, 8 bp, mouse
tccccgcc

SEQ ID NO: 3 - DNA, 30 bp, mouse
ttccccgccgttccccgccgttccccgccg

SEQ ID NO: 4 - DNA, 10 bp, mouse
cggcggggaa

SEQ ID NO: 5 - DNA, 30 bp, mouse
cggcggggaacggcggggaacggcggggaa

SEQ ID NO: 6 - DNA, 16 bp, mouse
tccccgcctccccgcc

SEQ ID NO: 7 - DNA, 8 bp, mouse
ggggggga

SEQ ID NO: 8 - DNA, 24 bp, mouse
gggggggagggggggaggggggga

SEQ ID NO: 9 - DNA, 8 bp, mouse
gccccgcc

SEQ ID NO: 10 - DNA, 8 bp, mouse
accccgcc

SEQ ID NO: 11 - DNA, 8 bp, mouse
tccccgtc

SEQ ID NO: 12 - DNA, 8 bp, mouse
tccccgct

SEQ ID NO: 13 - DNA, 8 bp, mouse
tccccacc

SEQ ID NO: 14 - DNA, 8 bp, mouse
tccccccc

SEQ ID NO: 15 - DNA, 8 bp, mouse
gacccgcc

SEQ ID NO: 16 - DNA, 8 bp, mouse
taaccgcc

SEQ ID NO: 17 - DNA, 8 bp, mouse
tcaacgcc

SEQ ID NO: 18 - DNA, 8 bp, mouse
tccaagcc

SEQ ID NO: 19 - DNA, 8 bp, mouse
tcccatcc

SEQ ID NO: 20 - DNA, 8 bp, mouse
tcccctac

SEQ ID NO: 21 - DNA, 8 bp, mouse
tccccgaa

SEQ ID NO: 22 - DNA, 8 bp, mouse
ggcgggga

SEQ ID NO: 23 - DNA, 92 bp, mouse
actcgcgccttaggaagcttgggtgtgtgtggcgcgctgtcttcccgctcgcgtcagggacctgcccgactcagcggctgccatggcatcag